(19) United States
(12) Patent Application Publication
(10) Pub. No.: US 2019/0088371 A1
(43) Pub. Date: Mar. 21, 2019

(54) NOVEL METHOD TO MEASURE NEURAL FORAMEN VOLUME OF THE SPINE

(71) Applicant: Regents of the University of Minnesota, Minneapolis, MN (US)

(72) Inventors: Charles Gerald Tan Ledonio, Minneapolis, MN (US); David Wayne Polly, Minneapolis, MN (US)

(21) Appl. No.: 16/134,389

(22) Filed: Sep. 18, 2018

Related U.S. Application Data
(60) Provisional application No. 62/560,409, filed on Sep. 19, 2017.

(51) Int. Cl. G16H 50/50; A61B 34/10

(52) U.S. Cl. CPC G16H 50/50; A61B 2034/104; A61B 2034/105; A61B 34/10

(57) ABSTRACT

A method includes creating a three-dimensional model of a bone structure that defines a space and placing a three-dimensional shape near the model of the bone structure. The three-dimensional shape is warped to surfaces of the bone structure to form a warped three-dimensional shape. A volume of the warped three-dimensional shape is determined to estimate a volume of the space.

### CROSS-REFERENCE TO RELATED APPLICATION

The present application is based on and claims the benefit of U.S. provisional application Ser. No. 62/560,409, filed Sep. 19, 2017, the content of which is hereby incorporated by reference in its entirety.

### BACKGROUND

The human spine consists of a column of vertebrae separated from each other by intervertebral discs. Between the vertebrae are intervertebral foramina, which are holes or openings that allow nerves to branch out from the spinal cord to various areas of the body. In foraminal stenosis, the volume of a foramen is reduced due to one or more of bone calcification, intervertebral disc deterioration and intervertebral disc bulging. Such foraminal stenosis can result in pressure being applied to the nerves exiting the foramen causing pain or loss of feeling.

The discussion above is merely provided for general background information and is not intended to be used as an aid in determining the scope of the claimed subject matter. The claimed subject matter is not limited to implementations that solve any or all disadvantages noted in the background.

### SUMMARY

A method includes creating a three-dimensional model of a bone structure that defines a space and placing a three-dimensional shape near the model of the bone structure. The three-dimensional shape is warped to surfaces of the bone structure to form a warped three-dimensional shape. A volume of the warped three-dimensional shape is determined to estimate a volume of the space.

In a further embodiment, a computing device has a processor executing instructions that cause the processor to perform steps of receiving a model of a region of a body; warping a three-dimensional shape to surfaces of the model to form a warped shape; and determining a volume of the warped shape.

In a still further embodiment, a method includes receiving a pre-procedure model of a region of a human body and a post-procedure model of the region. A first three-dimensional shape is formed to describe a space in the pre-procedure model and a second three-dimensional shape is formed to describe the space in the post-procedure model. The first three-dimensional shape and the second three-dimensional shape are used to determine a relative change in volume for the space between the pre-procedure model and the post-procedure model.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is a flow diagram of a method of determining a relative change in a foramen due to a spinal procedure.

FIG. 2 is a flow diagram for determining a volume of a foramen in accordance with one embodiment.

FIG. 3 is a block diagram of a system for determining a volume of a foramen.

FIG. 4 is a side view of a 3D model of a spinal column.

FIG. 5 is a perspective sectional view of the 3D model of FIG. 4

FIG. 6 is a top sectional view of the 3D model of FIG. 4.

FIG. 7 is a perspective view of a shaped volume in the form of an icosphere.

FIG. 8 is a top sectional view a 3D model of a spinal column with a three-dimensional shape positioned to encompass a foramen

FIG. 9 is a side view of the 3D model of FIG. 8

FIG. 10 is a top sectional view of the 3D model of FIG. 8 with the three-dimensional shape warped to the surface of the bone structures.

FIG. 11 is a side view of the 3D model of FIG. 10.

FIG. 12 is a perspective view of a warped 3D shaped volume.

FIG. 13 is a perspective view of the warped 3D shaped volume after subtraction of the 3D model of the spine.

FIG. 14 is a block diagram of a computing device that is used as either a client or server in accordance with the various embodiments.

### DETAILED DESCRIPTION OF ILLUSTRATIVE EMBODIMENTS

Diagnosing foraminal stenosis and evaluating the improvement in foraminal spacing after treatment has been difficult because of the irregular and inconsistent shapes of the bone structures forming the foramen. These irregularities make it difficult to consistently measure the spaces available for nerves to pass through the foramen.

In accordance with the various embodiments, a method and system are provided that generate a more descriptive value of the foraminal spacing by providing a measure of the volume between the bone structures at the foramen.

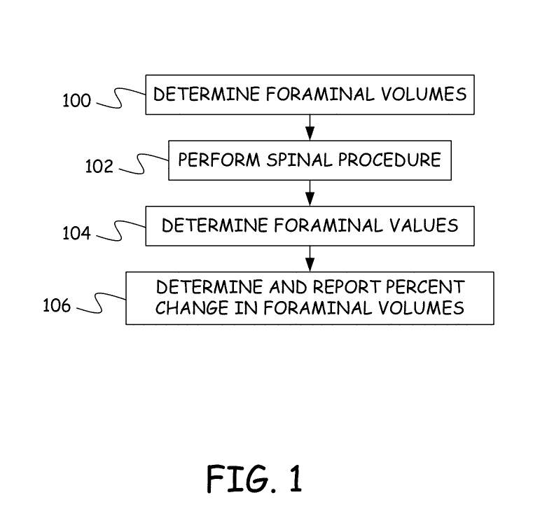

FIG. 1 provides a flow diagram of a method for evaluating a treatment of foraminal stenosis in accordance with one embodiment. In step 100 of FIG. 1, one or more foraminal volumes are determined as discussed further below. At step 102, a spinal procedure is performed to improve the foraminal spacing by for example placing a spacer between two vertebrae or removing portions of the vertebrae. At step 104, the foraminal volumes are determined once again as discussed further below and at step 106, the two foraminal volumes determined in step 100 and 104 are used to determine a percent change in foraminal volume. That percent change is then reported to indicate the effectiveness of the spinal procedure performed at step 102.

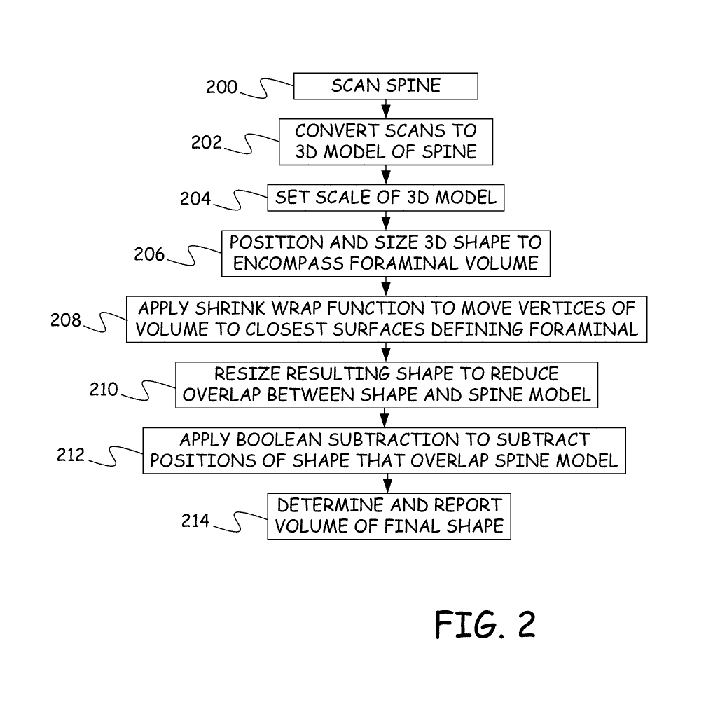

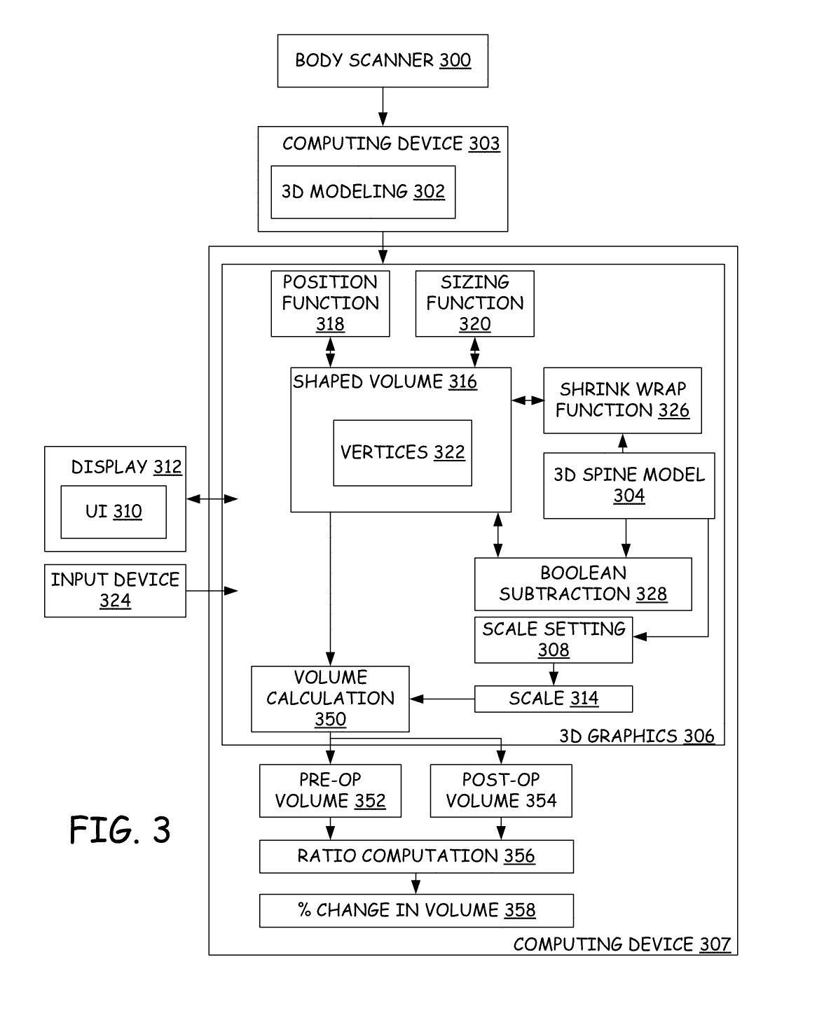

FIG. 2 provides a flow diagram of a method of determining foraminal volumes in accordance with one embodiment and FIG. 3 provides a block diagram of a system for determining such volumes and determining and reporting the effectiveness of a spinal procedure as found in FIG. 1. At step 200 of FIG. 2, a scan is performed of the spine or the portion of the spine suspected to contain a foraminal stenosis using a body scanner 300. In accordance with one embodiment, body scanner 300 is a computerized tomography (CT) scanner, which captures a plurality of x-rays of the spine at different viewing angles to produce a series of x-ray images. In other embodiments, body scanner 300 is a Magnetic Resonance Imaging (MRI) scanner, which produces a series of images representing slices of a portion of the body.

The series of x-ray images (for CT scanners) or the series of slice images (for MRI) is provided to 3D modeling software 302 operating on a computing device 303, which converts the series of images into a 3D model of the spine at step 202. This results in a 3D model of the spine 304, which is provided to a 3D graphics program 306 operating on a computing device 307. In accordance with one embodiment, the 3D model of the spine 304 is a model of just one section of the spine and does not model the entire spine. Further, the 3D model of the spine when constructed from a CT scan, typically only includes models of bone and cartilage in the spine and does not model soft tissues because such tissue does not appear in the x-ray images. In embodiments where the body scanner is an MRI scanner, some or all of the soft tissue in or near the spine can also be modeled in 3D model 304.

At step 204, a scale 314 of 3D model 304 is set using 3D graphics program 306. In particular, a scale setting module 308 generates one or more inputs on a user interface 310 shown on a display 312 for a user to designate the length of a portion of 3D model 304. In accordance with one embodiment, a physical marker with a known length is held outside of the body during scanning so that the physical marker is captured in the images and can be modeled in 3D model 304. The 3D model of the physical marker can then be used to set the scale 314 for 3D model 304 by assigning the known length of the physical marker to the corresponding dimension of the physical marker's 3D model. Alternatively, the scale for 3D model 304 can be set by setting an arbitrary distance for a dimension of the 3D model of one of the vertebrae. Which portion of the vertebrae is selected is immaterial as long as the same portion can be identified in the pre-procedure and post-procedure models of the spine. By assigning the same arbitrary distance to the same dimension in both the pre-procedure and post-procedure models of the spine, the scales of the two models are set equal to each other allowing for a meaningful comparison of the foraminal volumes in the two models.

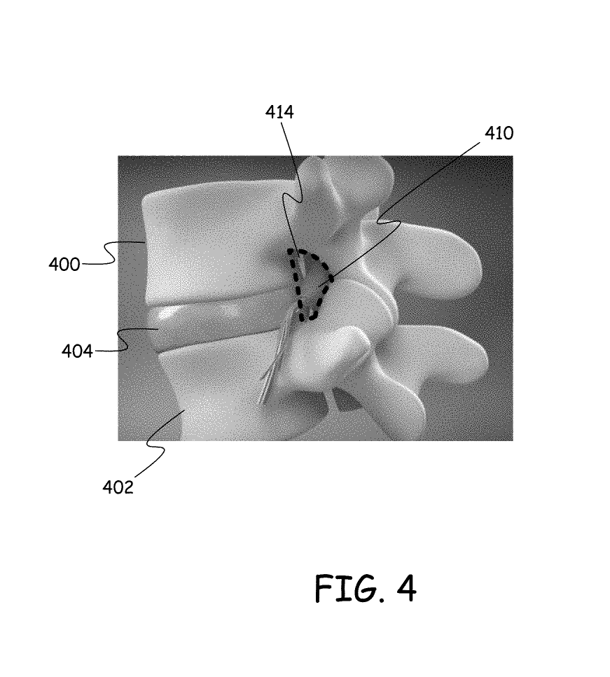

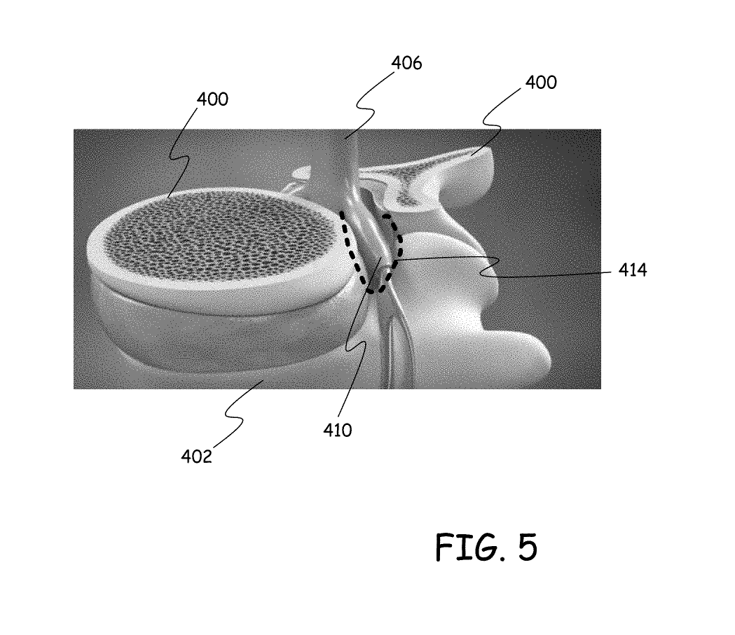

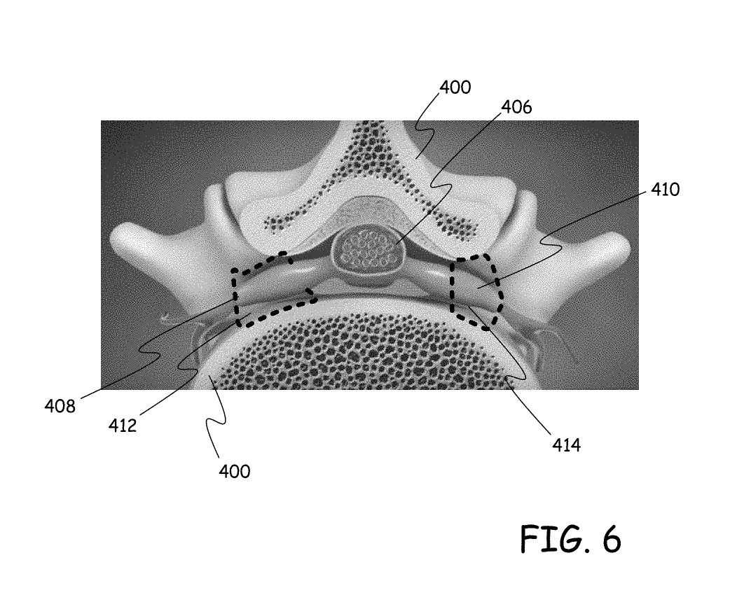

FIG. 4 provides a side view, FIG. 5 provides a sectional perspective view, and FIG. 6 provides a top sectional view of one example of 3D spine model 304 in which both bone and soft tissue is part of 3D model 304. As shown in FIGS. 4-6, 3D model 304 includes a model of a first vertebrae 400, a second vertebrae 402, a intervertebral disc 404 between vertebrae 400 and 402, spinal cord 406 and nerve roots 408 and 410. Nerve roots 408 and 410 extend through two respective intervertebral foramen 412 and 414, where each intervertebral foramen is a space or opening defined by the exterior surfaces of vertebrae 400 and 402 and intervertebral disc 404. The boundaries of the intervertebral foramen 412 and 414 are shown with dotted lines in FIGS. 4-6.

At step 206 of FIG. 2, a 3D shape or shaped volume 316 is positioned and sized using a position function 318 and a sizing function 320 so that the shaped volume 316 encompasses a foraminal volume or space. In accordance with one embodiment, shaped volume 316 is defined by a set of vertices 322 and positioning and sizing shaped volume 316 involves setting the positions of vertices 322. In accordance with one embodiment, position function 318 and sizing function 320 receive inputs from an input device 324 that are used to set the position and size of shaped volume 316 relative to 3D spine model 304.

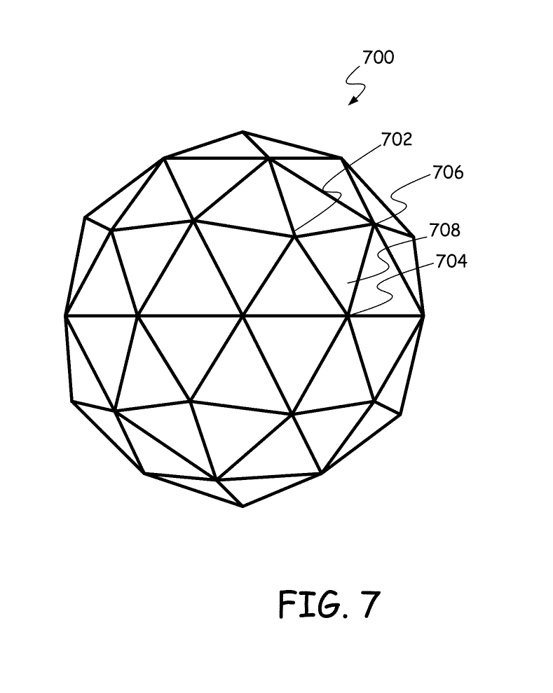

In accordance with one embodiment, shaped volume 316 is an icosphere, such as icosphere 700 of FIG. 7. Icosphere 700 is defined by a plurality of vertices, such as vertices 702, 704 and 706 where a set of vertices describes a polygon. For example, vertices 702, 704 and 706 describe a triangle 708 along the surface of icosphere 700. Icosphere 700 represents one example of a shaped volume 316 that may be used with the various embodiments. Those skilled in the art will recognize that other volumes may be used with the various embodiments. In many embodiments, shapes with larger numbers of vertices provide more accurate volume values for the foraminal spaces.

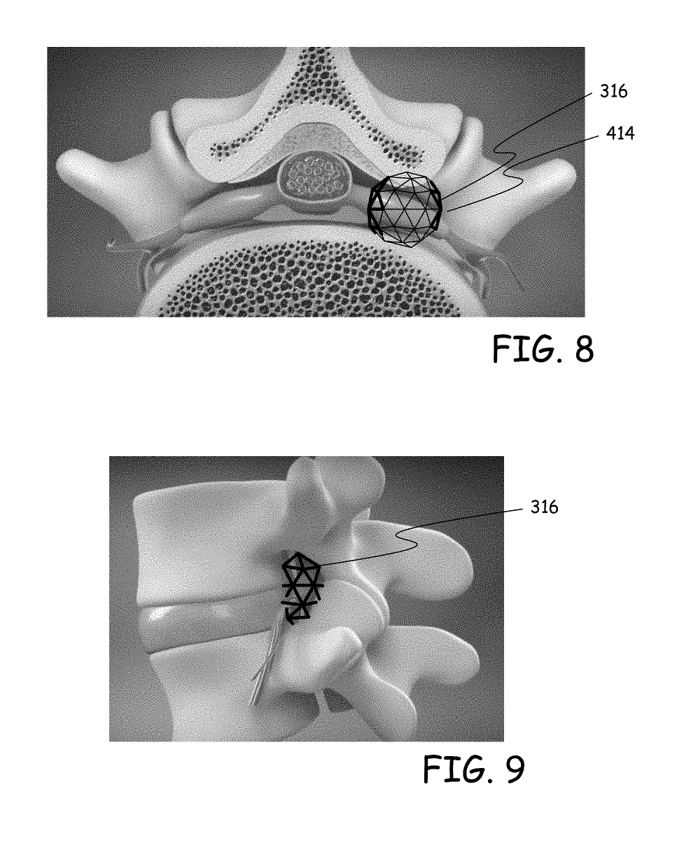

FIGS. 8 and 9 show shaped volume 316 in the form of an icosphere positioned to encompass intervertebral foramen 414. Thus, shaped volume 316 is positioned and sized such that the vertices of shaped volume 316 are located in the bone structures surrounding foramen 414 or extend outside of the foramen 414 in the interior of the spine or to the side of the spine. In FIG. 8, edges shown between vertices that are within a bone structure are shown with lighter weight lines than edges that are exterior to the foramen. In FIG. 9, only the edges that extend outside of the foramen are shown.

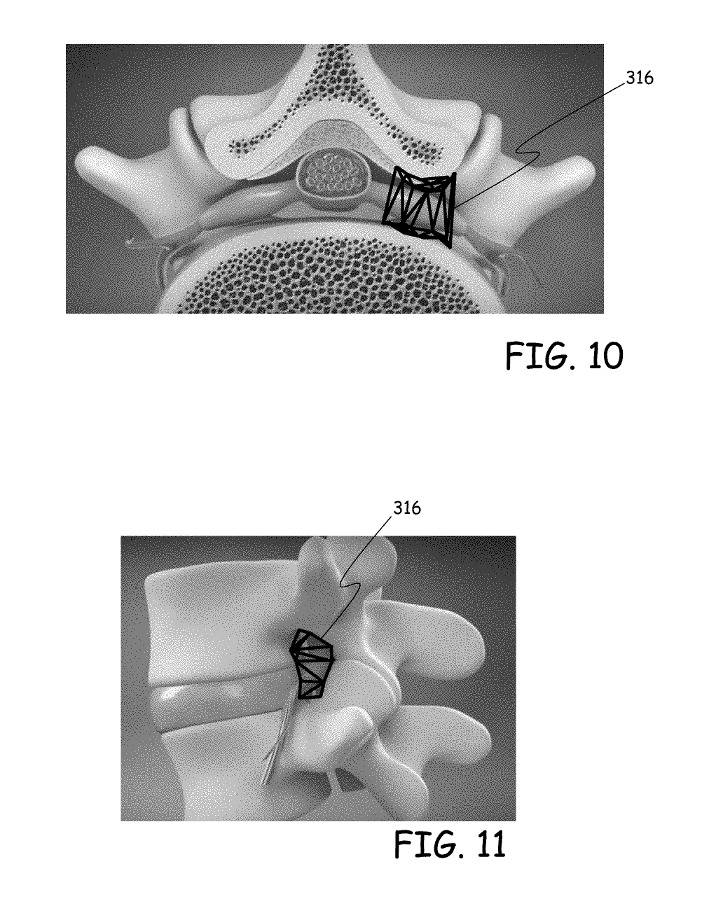

At step 208, an input from input device 324 activates shrink wrap function 326, which adjusts vertices 322 of shaped volume 316 so that each vertex moves to a respective point on the exterior surface of 3D spine model 304 that is closest to the vertex. FIGS. 10 and 11 show the appearance of shaped volume 316 after the shrink wrap function 326 has been applied showing that shaped volume 316 has taken on an irregular shape as opposed to the icosphere shape shown in FIGS. 8 and 9.

At step 210, sizing function 320 is used to resize the shrink wrap shape to reduce overlap between the shrink wrap shape and the 3D spine model. In accordance with one embodiment, this resizing is performed from inputs provided by input device 324 to sizing function 320. During resizing, the location of the vertices is monitored on display 312 so that no space is created between the bone structures that define the foramen and the resized shaped volume 316.

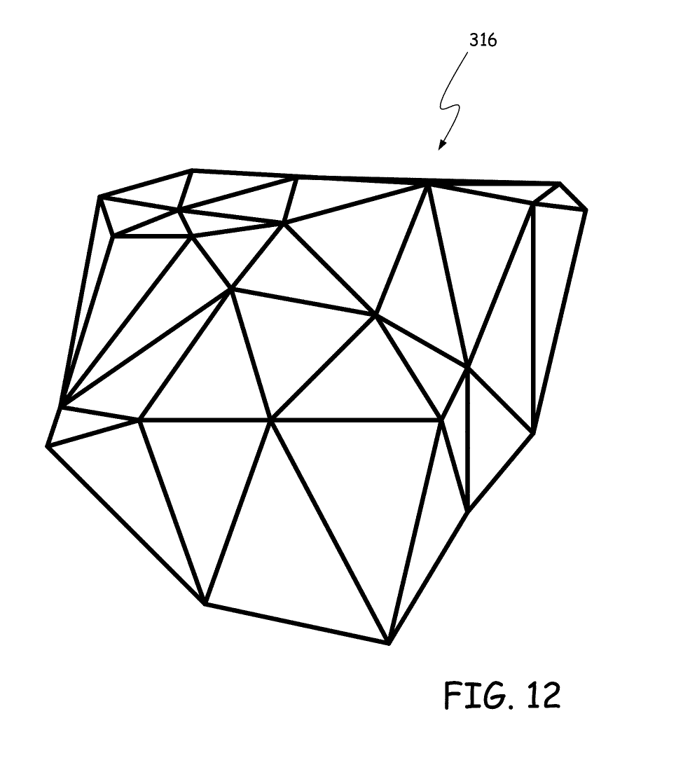

FIG. 12 provides a perspective view of the shrink wrapped and resized shaped volume 316 showing the irregular shape generated by shrink wrap function 326. The irregularity of shaped volume 316 is indicative of the challenge in defining the foraminal space since this irregular volume varies for each foraminal space and cannot be predicted.

At step 212, a Boolean subtraction 328 is applied to shaped volume 316 using 3D spine model 304. In particular, 3D spine model 304 is subtracted from shaped volume 316, such that portions of shaped volume 316 that overlap with 3D spine model 304 are redefined to follow the contours of 3D spine model 304. In accordance with one embodiment, this is performed by identifying where 3D spine model 304 overlaps shaped volume 316. For each separate overlap, all of the vertices of the vertices of shaped volume 316 that are within 3D spine model 304 are replace with the vertices of 3D spine model 304.

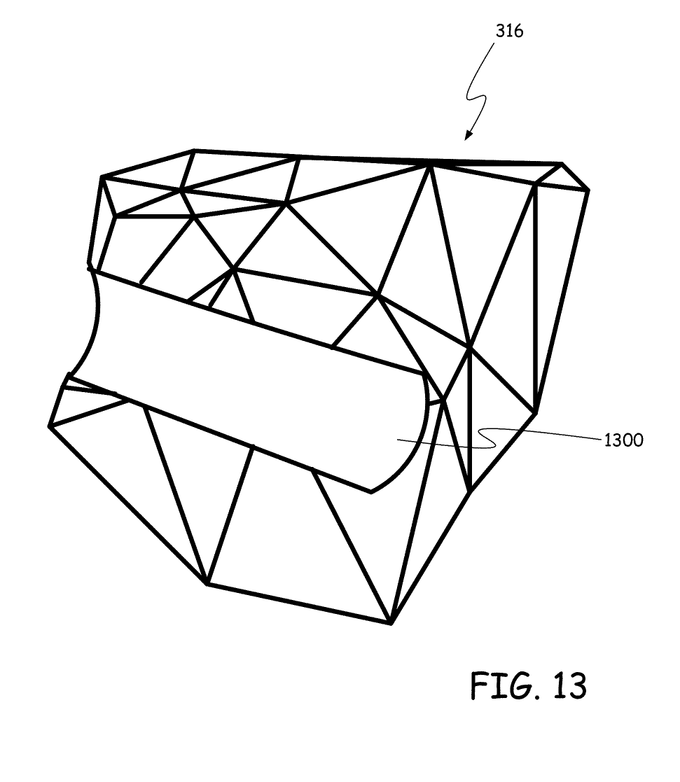

FIG. 13 provides an example of shaped volume 316 after Boolean subtraction 328 has been performed in one embodiment. As shown, Boolean subtraction 328 forms a shaped surface 1300 where 3D spine model 304 intersected the previous version of shaped volume 316 shown in FIG. 12.

At step 214, a volume calculation module 350 uses the vertices 322 of shaped volume 316 created in step 212 and the scale 314 set in step 204 to calculate the volume of shaped volume 316. When the volume is being determined before the spinal operation in step 100 of FIG. 1, the volume is saved as pre-op volume 352. When the volume is being determined after the spinal procedure at step 104, the volume is saved as post-op volume 354. In step 106 of FIG. 1, a ratio computation module 356 uses the pre-op volume 352 and the post-op volume 354 to determine a percent change in volume 358 that is reported to the user through user interface 310 on display 312. The percent change in volume 358 is the ratio of post-op volume 354 to pre-op volume 352 in some embodiments. In other embodiments, the percent change in volume 358 is computed as the difference between post-op volume 354 and pre-op volume 352 divided by pre-op volume 352.

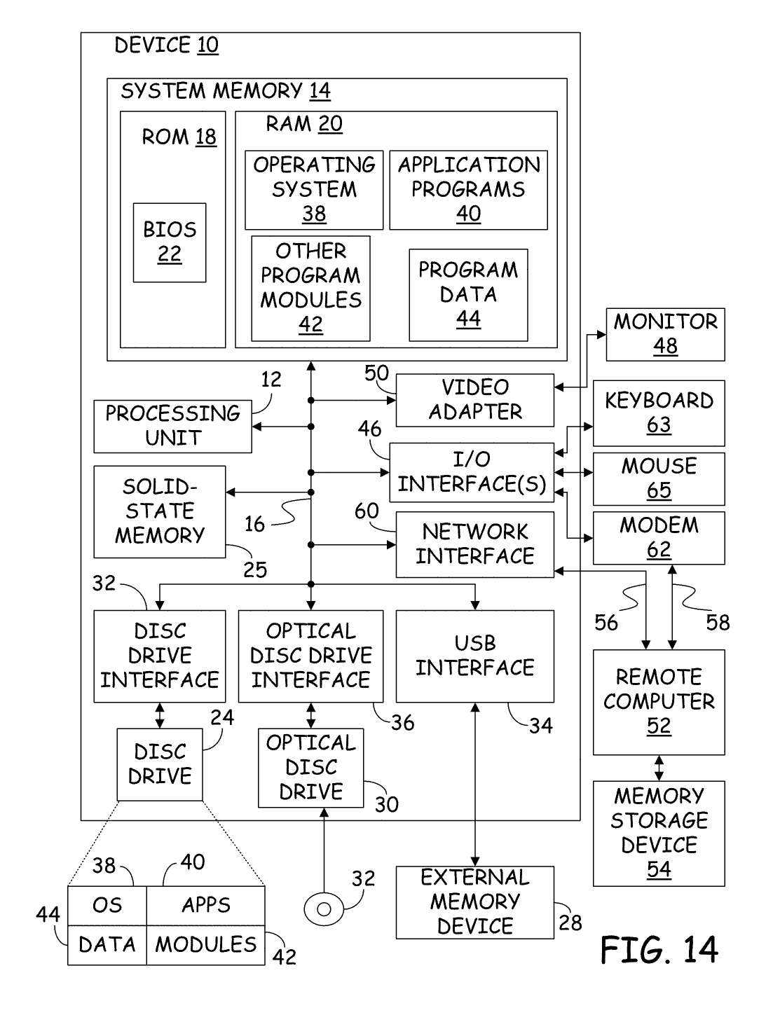

FIG. 14 provides an example of a computing device 10 that can be used as a server device or client device in the embodiments above. Computing device 10 includes a processing unit 12, a system memory 14 and a system bus 16 that couples the system memory 14 to the processing unit 12. System memory 14 includes read only memory (ROM) 18 and random access memory (RAM) 20. A basic input/output system 22 (BIOS), containing the basic routines that help to transfer information between elements within the computing device 10, is stored in ROM 18. Computer-executable instructions that are to be executed by processing unit 12 may be stored in random access memory 20 before being executed.

Embodiments of the present invention can be applied in the context of computer systems other than computing device 10. Other appropriate computer systems include handheld devices, multi-processor systems, various consumer electronic devices, mainframe computers, and the like. Those skilled in the art will also appreciate that embodiments can also be applied within computer systems wherein tasks are performed by remote processing devices that are linked through a communications network (e.g., communication utilizing Internet or web-based software systems). For example, program modules may be located in either local or remote memory storage devices or simultaneously in both local and remote memory storage devices. Similarly, any storage of data associated with embodiments of the present invention may be accomplished utilizing either local or remote storage devices, or simultaneously utilizing both local and remote storage devices.

Computing device 10 further includes an optional hard disc drive 24, an optional external memory device 28, and an optional optical disc drive 30. External memory device 28 can include an external disc drive or solid state memory that may be attached to computing device 10 through an interface such as Universal Serial Bus interface 34, which is connected to system bus 16. Optical disc drive 30 can illustratively be utilized for reading data from (or writing data to) optical media, such as a CD-ROM disc 32. Hard disc drive 24 and optical disc drive 30 are connected to the system bus 16 by a hard disc drive interface 32 and an optical disc drive interface 36, respectively. The drives and external memory devices and their associated computer-readable media provide nonvolatile storage media for the computing device 10 on which computer-executable instructions and computer-readable data structures may be stored. Other types of media that are readable by a computer may also be used in the exemplary operation environment.

A number of program modules may be stored in the drives and RAM 20, including an operating system 38, one or more application programs 40, other program modules 42 and program data 44. In particular, application programs 40 can include programs for implementing any one of 3D graphics program 306, 3D modeling 302, and ratio computation 356, for example. Program data 44 may include data such as 3D spine model 304, scale 314, pre-op volume 352, post-op volume 354, and percent change in volume 358, for example.

Processing unit 12, also referred to as a processor, executes programs in system memory 14 and solid state memory 25 to perform the methods described above.

Input devices including a keyboard 63 and a mouse 65 are optionally connected to system bus 16 through an Input/Output interface 46 that is coupled to system bus 16. Monitor or display 48 is connected to the system bus 16 through a video adapter 50 and provides graphical images to users. Other peripheral output devices (e.g., speakers or printers) could also be included but have not been illustrated. In accordance with some embodiments, monitor 48 comprises a touch screen that both displays input and provides locations on the screen where the user is contacting the screen.

The computing device 10 may operate in a network environment utilizing connections to one or more remote computers, such as a remote computer 52. The remote computer 52 may be a server, a router, a peer device, or other common network node. Remote computer 52 may include many or all of the features and elements described in relation to computing device 10, although only a memory storage device 54 has been illustrated in FIG. 14. The network connections depicted in FIG. 14 include a local area network (LAN) 56 and a wide area network (WAN) 58. Such network environments are commonplace in the art.

The computing device 10 is connected to the LAN 56 through a network interface 60. The computing device 10 is also connected to WAN 58 and includes a modem 62 for establishing communications over the WAN 58. The modem 62, which may be internal or external, is connected to the system bus 16 via the I/O interface 46.

In a networked environment, program modules depicted relative to the computing device 10, or portions thereof, may be stored in the remote memory storage device 54. For example, application programs may be stored utilizing memory storage device 54. In addition, data associated with an application program may illustratively be stored within memory storage device 54. It will be appreciated that the network connections shown in FIG. 14 are exemplary and other means for establishing a communications link between the computers, such as a wireless interface communications link, may be used.

Although elements have been shown or described as separate embodiments above, portions of each embodiment may be combined with all or part of other embodiments described above.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms for implementing the claims.

Although the present invention has been described with reference to preferred embodiments, workers skilled in the art will recognize that changes may be made in form and detail without departing from the spirit and scope of the invention.

What is claimed is:

1. A method comprising:
creating a three-dimensional model of a bone structure that defines a space;
placing a three-dimensional shape near the model of the bone structure;
warping the three-dimensional shape to surfaces of the bone structure to form a warped three-dimensional shape; and
determining a volume of the warped three-dimensional shape to estimate a volume of the space.

2. The method of claim 1 wherein warping the three-dimensional shape comprises shifting vertices of the three-dimensional shape to surfaces of the bone structure.

3. The method of claim 2 wherein shifting vertices of the three-dimensional shape forms a shrink-wrapped shape and wherein warping the three-dimensional shape further comprises subtracting a portion of the bone structure from the shrink-wrapped shape to form the warped three-dimensional shape.

4. The method of claim 1 wherein placing the three-dimensional shape near the bone structure comprises placing the three-dimensional shape so that the three-dimensional shape encompasses the space.

5. The method of claim 1 further comprising:
creating a three-dimensional post-procedure model of the bone structure after a spinal procedure, where the bone structure after the spinal procedure defines a modified space;
placing a second three-dimensional shape near the post-procedure model of the bone structure;
warping the second three-dimensional shape to surfaces of the post-procedure model of the bone structure to form a second warped three-dimensional shape; and
determining a volume of the second warped three-dimensional shape to estimate a volume of the modified space.

6. The method of claim 5 further comprising determining a ratio using the volume of the warped three-dimensional shape and the volume of the second warped three-dimensional shape.

7. The method of claim 1 wherein determining the volume of the second warped three-dimensional shape comprises using a scale set for the model of the bone structure and a second scale set for the post-procedure model of the bone structure.

8. A computing device comprising:
a processor executing instructions that cause the processor to perform steps comprising:
receiving a model of a region of a body;
warping a three-dimensional shape to surfaces of the model to form a warped shape; and
determining a volume of the warped shape.

9. The computing device of claim 8 wherein warping the three-dimensional shape comprises shifting vertices of the three-dimensional shape to the surfaces of the model to form a shrink-wrapped shape.

10. The computing device of claim 9 wherein warping the three-dimensional shape further comprises subtracting the model of the region of the body from the shrink-wrapped shape.

11. The computing device of claim 8 wherein the steps performed by the processor further comprise:
receiving a post-procedure model of the region of the body formed after a medical procedure is performed on the region;
warping a second three-dimensional shape to surfaces of the post-procedure model to form a second warped shape; and
determining a volume of the second warped shape.

12. The computing device of claim 11 wherein the steps performed by the processor further comprises:
determining a ratio using the volume of the warped shape and the volume of the second shape.

13. The computing device of claim 11 wherein determining a volume of the second warped shape comprises setting a scale for the post-procedure model of the region.

14. The computing device of claim 13 wherein setting a scale for the post-procedure model comprises setting a value for a distance between two points in the post-procedure model to be equal to a value set for the distance between the two points in the model of the region.

15. A method comprising:
receiving a pre-procedure model of a region of a human body and a post-procedure model of the region;
forming a first three-dimensional shape to describe a space in the pre-procedure model;
forming a second three-dimensional shape to describe the space in the post-procedure model; and
using the first three-dimensional shape and the second three-dimensional shape to determine a relative change in volume for the space between the pre-procedure model and the post-procedure model.

16. The method of claim 15 wherein forming the first three-dimensional shape comprises placing an initial three-dimensional shape so that the three-dimensional shape encompasses the space then deforming the initial three-dimensional shape to form the first three-dimensional shape.

17. The method of claim 16 wherein deforming the initial three-dimensional shape comprises shifting every vertex of the initial three-dimensional shape to a surface of the pre-procedure model.

18. The method of claim 17 wherein shifting every vertex of the initial three-dimensional shape to a surface of the pre-procedure model forms a shrink-wrapped shape and wherein deforming the initial three-dimensional shape further comprises subtracting the pre-procedural model from the shrink-wrapped shape to form the first three-dimensional shape.

19. The method of claim 15 wherein using the first three-dimensional shape and the second three-dimensional shape to determine a relative change in volume for the space between the pre-procedure model and the post-procedure model comprises determining at least one scaling factor to scale the first three-dimensional shape to the second three-dimensional shape.

20. The method of claim 16 wherein the initial three-dimensional shape comprises an icosphere.
